(19) United States
(12) Patent Application Publication
(10) Pub. No.: US 2017/0028098 A1
(43) Pub. Date: Feb. 2, 2017

(54) SKIN COMPATIBLE CURING ADHESIVES FOR ADHERING DEVICES TO MAMMALIAN BODY

(71) Applicant: Trio Healthcare Ltd, North Yorkshire (GB)

(72) Inventors: Lloyd PEARCE, North Yorkshire (GB); Mahesh SAMBASIVAM, Pennington, NJ (US)

(73) Assignee: Trio Healthcare Ltd, North Yorkshire (GB)

(21) Appl. No.: 15/100,238

(22) PCT Filed: Nov. 24, 2014
(86) PCT No.: PCT/GB2014/053460

§ 371 Date: May 27, 2016

Related U.S. Application Data
(60) Provisional application No. 61/911,745, filed on Dec. 4, 2013.

(30) Foreign Application Priority Data
Apr. 2, 2014 (GB) 1405947.1

(51) Int. Cl. A61L 24/04

(52) U.S. Cl. CPC A61L 24/046

(57) ABSTRACT

Skin compatible curing adhesives for adhering devices or appliances to mammalian body are disclosed. Adhesive compositions to protect per-anal, peri-stomal, peri-wound, and peri-fistula skin are disclosed. The skin compatible curable adhesive includes at least one silylated polymer, silylated macromer, silylated monomer, or a combination thereof; at least one curing catalyst; and at least one crosslinking agent.

### BACKGROUND

Technical Field

The present disclosure relates to skin compatible curing adhesives for protecting skin and/or for adhering medical devices to mammalian body.

Background

There are medical conditions such as ostomy, pressure ulcer, fistula, chronic and acute wounds, highly exuding wounds, and fecal incontinence that require management of the waste outputted by the body. Management of such waste is critical in improving the condition such as related to wound healing, and maintaining a quality of life in the case of ostomy and fecal incontinence. Devices or appliances used to manage the above conditions are secured to the body using adhesives which are part of the device or appliance.

In the case of ostomy, the collection bag and adhesive wafer, either as separate components (referred to as “2-piece system”) or permanently jointed together (referred to as “1-piece system”) is attached to the peristomal skin through the adhesive wafer to manage stomal waste. It is challenging to securely attach an ostomy device or appliance to an abdominal stoma due to anatomical contour, skin folds or creases, irregular-shaped stomas, surgical scars, etc. These devices work well for some people, whereas for a majority of ostomates, additional accessories such as an adhesive paste are required to protect the peristomal skin from contact with the stomal waste or effluent. The adhesive paste in this case acts as a sealant, a gasket, or a dam around the peristomal skin, and facilities better anchorage of the ostomy appliance to the body. The adhesive paste conforms to the anatomy, fills the gap between the appliance and the stoma, and adheres securely to skin and to the adhesive wafer.

The commercially available ostomy adhesive pastes are based on blends of non-curing hydrophobic and/or hydrophilic polymers with plasticizers, solvents, and fillers such as hydrocolloids.

In the case of wound care, dressings are used to manage the exudate and to promote wound healing. Wounds can occur in any part of the body, and depending on the location, it is challenging to adhere a dressing to the wound. Similar situation arises in fistula, perianal skin management, fecal incontinence, where the anatomy of the body renders it difficult to securely adhere or attach devices to manage the exudate.

### SUMMARY

One objective of this disclosure is to provide skin-compatible curing adhesives for adhering medical devices to a mammalian body.

Another objective is to provide skin-compatible curing adhesives to protect a peri-skin surface from one or more bodily fluids.

The above objectives are wholly or partially met by devices, articles, appliances, intermediates, compounds, and methods according to the appended claims. Features and aspects are set forth in the appended claims, and in the following description in accordance with the present disclosure.

According to a first aspect there is provided a skin compatible curable adhesive to protect a region of a skin surface, for the treatment or management of one or more medical conditions, the adhesive including at least one silylated polymer, silylated macromer, silylated monomer, or a combination thereof, at least one curing catalyst, and at least one crosslinking agent.

In aspects, the adhesive may include a pre-cured state and a post-cured state. In aspects, the post-cured state may be tacky to the touch with a loop tack force greater than 0.1 N/in. In aspects, the post-cured state may be non-tacky to the touch with a loop tack force of less than 0.1 N/in. In aspects, the adhesive may be configured to form a cohesive bond to a surface of a device applied thereto only while in the pre-cured state.

In aspects, a skin compatible curable adhesive may include a pre-cured form having a viscosity less than 500,000 cps, less than 250,000 cps, or less than 100,000 cps, or the like.

In aspects, the adhesive may be configured to be applied to the region of skin in the pre-cured state, and to transition to the post cured state on the skin. In aspects, the adhesive in the post-cured state may be configured to form a bond to the skin, the peel strength thereof in the range of 0.1 to 10 N/in. In aspects, the adhesive may be configured to transition from the pre-cured state to the post-cured state upon application to the skin, the transition taking less than 7 days, less than 24 hours, less than 1 hour, less than 30 minutes, less than 15 minutes, or the like.

In aspects, the transition may be facilitated by the adhesive undergoing a curing reaction to transition between the pre-cured state and the post-cured state, the curing reaction initiated via exposure of the adhesive to moisture, ultraviolet light, visible light, infrared radiation, heat, oxygen, a combination thereof, or the like.

In aspects, the silylated polymer, macromer, or monomer may include two or more pendant or terminal silylated functional groups. Some non-limiting examples of such groups include vinyl-silyl, hydride-silyl, alkoxysilyl, aryloxysilyl, acryloxysilyl, alkyloximinosilyl, hydroxyl-silyl, amino-silyl, halo-silyl, combinations thereof, and the like.

In aspects, the adhesive may include one or more of divinyl-terminated polydimethylsiloxane, dihydride-terminated polydimethylsiloxane, polymethylhydrogensiloxane-polydmethylsiloxane copolymer, acetoxy-terminated polydimethylsiloxane, silylated polyether, silylated polyurethane, silylated polyolefin, silylated polyester, silylated acrylic, combinations thereof, or the like.

In aspects, an adhesive in accordance with the present disclosure may include a silicone polymer, macromer, monomer, or a combination thereof.

In aspects, the adhesive may include one or more hydrophilic additive. Some non-limiting examples of such additives include sodium carboxymethylcellulose (NaCMC), pectin, gelatin, chitosan, polyvinyl pyrrolidone and its copolymers, polyvinyl alcohol and its copolymers, polyacrylic acid, its copolymers and salts, nanoclays, cellulosic fibers, starch, polyacrylamide and its copolymers, poly(N-alkylacrylamide) and its copolymers, polyethylene glycol and its copolymers, polyethyleneoxide, unmodified and modified silica, poly(maleic anhydride) and its copolymers, combinations thereof, and the like. Other hydrophilic additives include hydrocolloid powders, ethylcellulose, hydroxyethylcellulose, hydropropylcellulose, hydroxypropylmethylcellulose, guar gum, xanthan gum, and the like.

In aspects, the catalyst may be at least partially tin-based, titanium-based, zinc-based, or copper-based, platinum based, or the like.

In aspects, the adhesive in accordance with the present disclosure may include one Or more of dibutyltindilaurate, bis(2-ethylhexanoate)tin, dioctyltindilaurate, tetraoctyltitanate, titanium diisopropoxide(bis-2,4-pentanedionate), titanium ethylhexyloxide, titanium(IV) tert-butoxide, tetrakis(trimethylsiloxy)titanium, platinum-divinyltetramethyldisiloxane complex, platinum -cylcovinylmethylsiloxane complex, combinations thereof, or the like.

In aspects, the crosslinking agent may include ethyltriacetoxysilane, methyltriacetoxysilane, vinyltriacetoxysilane, tetraethoxysilane, methyltrimethoxysilane, methyltris(methylethylketoximino)silane, b is(N-methybenzam ido)ethoxymethyl-si lane, vinyl-MQ resin, hydride-MQ resin, hydroxyl-MQ resin, polymethylhydrogensiloxane-dimethylsiloxane copolymer, polymethylvinylsiloxane-dimethylsiloxane copolymer, combinations thereof, or the like.

Optionally, the adhesive composition comprises a silylated polymer at 80 to 98% by weight; at least one curing catalyst at trace to 4% by weight; and at least one crosslinking agent at trace to 15% by weight. More preferably, the adhesive composition comprises a silylated polymer at 90 to 96% by weight; at least one curing catalyst at trace to 2.0% by weight; and at least one crosslinking agent at 1.0 to 3.5% by weight. Optionally, the silylated polymer comprises a combination of a first type of silylated polymer and a second type of silylated polymer. Optionally, the first silylated polymer may be included at 40 to 60% by weight and the second silylated polymer may be included at 40 to 60% by weight. Optionally, the first and second silylated polymers are hydroxyl-terminated polysiloxanes. Optionally, the adhesive composition comprises 1 to 10% by weight of a filler such as a silica and in particular a hydrophobic fumed silica and/or a hydrophilic additive. Optionally, the filler is included at 1 to 6% by weight. Optionally, the adhesive composition comprises a silylated polymer at 80 to 90% by weight; at least one curing catalyst at trace to 2% by weight; and at least one crosslinking agent at 5 to 15% by weight. More preferably, the adhesive composition comprises a silylated polymer at 82 to 89% by weight; at least one curing catalyst at trace to 5% by weight; and at least one crosslinking agent at 6 to 10% by weight. Optionally, the adhesive composition comprises a first type of crosslinking agent at 6 to 10% by weight and a second type of crosslinking agent at trace to 2% by weight. Optionally, the adhesive composition further comprises a hydrophilic additive, pectin. Optionally, the pectin is included at trace to 5% by weight and more preferably 0.5 to 3% by weight.

In aspects, the post-cured state of the adhesive may be substantially insoluble in water, bodily fluids, common cleaning materials, etc.

In aspects, the adhesive may be configured to be stored as a single component system.

In aspects, the adhesive may be configured to be stored as a two-part system, the two-parts mixable to form a pre-cured state in accordance with the present disclosure.

In aspects, the region of skin may be a peri-anal, peri-stomal, peri-wound, peri-fistula skin, or the like.

According to a further aspect there is provided, use of a skin compatible curable adhesive in accordance with the present disclosure disclosure to protect peri-anal, peri-stomal, peri-wound, or peri-fistula skin.

According to a further aspect there is provided a method for protecting and/or managing a peri-skin surface on a body including administering an adhesive composition to the peri-skin surface, the adhesive includes at least one silylated polymer, silylated macromer, silylated monomer, or a combination thereof; at least one curing catalyst; and at least one crosslinking agent; applying a device to the adhesive composition, and curing the adhesive composition.

In aspects, the method may include applying a primer to, cleaning, and/or preparing the peri-skin prior to the administering of the adhesive composition.

In aspects, the method may include mixing two or more parts to form the adhesive composition prior to the step of administering the adhesive composition to the peri-skin.

In aspects, the mixing may be performed with a static mixer, the two or more parts stored in compartments, and delivered to the peri-skin via the static mixer.

In aspects, the method may include forming a skin-side bond between the peri-skin and the adhesive composition. In aspects, the peel strength of the skin-side bond may be in the range of 0.1 to 10 N/in, or the like.

In aspects, the method may include forming a device-side bond between the device and the adhesive composition. In aspects, the peel strength of the device-side bond may be greater than 15 g/in, greater than 125 g/in, greater than 225 g/in, or the like.

The method may include maintaining the adhesive composition against the peri-skin for greater than 24 hours, greater than 48 hours, greater than 72 hours.

The method may include simultaneously removing the device and the adhesive composition from the peri-skin. In aspects, the cohesive bond formed between the device and the adhesive composition may be maintained after removed from the peri-skin.

In aspects, the method may include administering the adhesive composition in a ring-like shape onto the peri-skin.

In aspects, the peri-skin may include a peri-anal, peri-stomal, peri-wound, peri-fistula skin, or the like.

In aspects, the device may include comprise a wound dressing, a surgical drape, a film, a foam, an ostomy adhesive wafer, an ostomy bag, or the like.

### DETAILED DESCRIPTION

Particular embodiments of the present disclosure are described herein below; however, the disclosed embodiments are merely examples of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

A skin compatible curable adhesive, in accordance with the present disclosure may be used to protect peri-anal, peri-stomal, peri-wound, and peri-fistula skin, for the treatment or management of a range of medical conditions (e.g. ostomy management, wound care, fistula management, etc.) may include at least one silylated polymer, macromer, monomer, or combination thereof, at least one curing catalyst, and at least one crosslinking agent.

In aspects, the adhesive may be secured to the peri-skin of the mammalian body by applying the adhesive (i.e. such as squeezed out of a tube, etc.), curing the adhesive in place to adhere to skin, attaching a device or appliance to the curing adhesive, and after use, removing the device or appliance from the peri-skin as one piece with the cured adhesive or as separate components, whereby the cured adhesive is essentially removed as one cohesive piece with minimal residue left behind on the peri-skin.

The term ‘curable adhesive’ encompasses an adhesive capable of forming a crosslinked polymer network or gel triggered by solvent evaporation, visible light, moisture, heat, infra-red radiation, ultra-violet radiation, or other known methods to initiate crosslinking reactions. The crosslinking reaction generally results in a polymer network with sufficient cohesive strength such that it can adhere to the peri-skin and to a device or appliance with sufficient bond strength and does not dissolve, or swell excessively in the presence of water, bodily waste, or other cleaning agents. Excessive swelling may be defined as weight gain due to absorption of fluid by 75%, more than 100%, more than 150%, or the like in the presence of a potential application-specific solvating agent (e.g. water, bodily waste, cleaning agent, etc.).

In aspects, the adhesive may be provided as a 1-part, a 2-part, or a multi-part curable system prior to application to the user. In aspects relating to a 1-part curable system, a user would apply it to the skin or peri-skin as provided and the adhesive would cure in place. In aspects relating to a 2-part, or a multi-part system, the parts may be provided as separate units (i.e. such as stored in separate compartments of a dispenser, within different compartments of a flat pack, etc.). Before, upon, and/or during administration of the parts to a skin surface, the parts may be mixed together (e.g. mixed manually, compartment mixed, via a static mixing nozzle, via a metered mixer, etc.). In aspects, the mixing may occur as the parts are transferred from respective storage compartments to the skin (i.e. within an application tool, within a dispenser, etc.).

A skin-compatible curable adhesive in accordance with the present disclosure, may include at least one silylated-functional polymer, macromer, and/or monomer, a curing catalyst, and a crosslinking agent, wherein the adhesive may be delivered to skin via a syringe, applicator, swab, tube, squeegee, dispenser, spatula, or other user friendly methods of applying a liquid or paste to human body.

An adhesive in accordance with the present disclosure may include curable 1-part or 2-part silicone adhesives, such as room temperature vulcanizable (RTV) silicones, or the like. These adhesives may substantially cure in the presence of moisture via condensation mechanism. Non-limiting examples of such adhesives are MED-1031, and MED-1037, both from Nusil Technology, LLC.

An adhesive in accordance with the present disclosure may include one or more addition curable silicone adhesives that are provided as a 2-part adhesive system. Non-limiting examples of such 2-part adhesive systems are A-103 Medical grade elastomer from Factor II, Incorporated and SILASTIC MDX4-4210 from Dow Corning Corporation. Such systems may cure to form a polymer network via hydrosilation addition reaction mechanism in the presence of a platinum catalyst.

The silylated polymer, silylated macromer, silylated oligomer, and/or silylated monomer may include at least two or more pendant or terminal silylated functional groups selected from vinyl-silyl, hydride-silyl, alkoxysilyl, aryloxysilyl, acryloxysilyl, alkyloximinosilyl, hydroxyl-silyl, amino-silyl, halo-silyl and combinations thereof. Examples of such curing polymers and oligomers are divinyl-terminated polydimethylsiloxane, dihydride-terminated polydimethylsiloxane, polymethylhydrogensiloxane-polydmethylsiloxane copolymer, hydroxyl-terminated polydimethylsiloxane and its copolymers, acetoxy-terminated polydimethylsiloxane, silylated polyethers such as “MS polymer”, available from Kaneka Corporation, silylated polyurethanes, silylated polyolefins, silylated polyesters, silylated acrylics, combinations thereof, and the like.

In aspects related to a skin compatible curable adhesive composition in accordance with the present disclosure, the curing catalyst for such curing reactions may be one or more of tin-based, titanium-based, zinc-based, or copper-based when the reaction is a condensation reaction. Non-limiting examples of tin catalysts are dibutyltindilaurate, bis(2-ethylhexanoate)tin, and dioctyltindilaurate. Non-limiting examples of titanium catalysts are tetraoctyltitanate, titanium ethylhexyloxide, titanium(IV) tert-butoxide, titanium diisopropoxide(bis-2,4-pentanedionate), and tetrakis(trimethylsiloxy)titanium. For addition curing reactions, the catalyst may be a platinum-based catalyst. Non-limiting examples of platinum catalysts for addition cure reactions are platinum-divinyltetramethyldisiloxane complex, and platinum-cylcovinylmethylsiloxane complex.

In aspects relating to a skin compatible curable adhesive composition in accordance with the present disclosure, one or more of the crosslinking agents for such compositions may include ethyltriacetoxysilane, methyltriacetoxysilane, vinyltriacetoxysilane, tetraethoxysilane, methyltris(methylethylketoximino)silane, bis(N-methybenzamido)ethoxymethyl-silane, vinyl-MQ resin, hydride-MQ resin, hydroxyl-MQ resin, methyltrimethoxysilane, polymethylhydrogensiloxane-dimethylsiloxane copolymer, polymethylvinylsiloxane-dimethylsiloxane copolymer, combinations thereof, and the like.

By device or appliance, used interchangeably, is meant an ostomy adhesive wafer, an ostomy bag, a fecal management bag, a wound dressing, a negative pressure wound therapy dressing or system, a fistula drain system, a peri-anal drain system, and a fecal incontinence drain system.

In aspects, the adhesive in accordance with the present disclosure may include one or more skin friendly additives to prevent or limit skin maceration, to maintain healthy skin, and/or to absorb moisture. Such additives may include but are not limited to hydrocolloids, hydrophilic polymers, hydrophilic additives, inorganic fillers, fibers, and combinations thereof. Some examples of such additives are but not limited to sodium carboxymethylcellulose (NaCMC), pectin, gelatin, chitosan, polyvinyl pyrrolidone and its copolymers, polyvinyl alcohol and its copolymers, polyacrylic acid, its copolymers and salts, nanoclays, cellulosic fibers, polyacrylamide and its copolymers, poly(N-alkylacrylamide) and its copolymers, polyethylene glycol and its copolymers, polyethyleneoxide, unmodified and modified silica, poly(maleic anhydride) and its copolymers, or combinations thereof.

In aspects, an adhesive in accordance with the present disclosure may include one or more solvents, which upon application of the adhesive composition to a skin surface may evaporate leaving a curable paste on the skin. Suitable skin-friendly solvents include but are not limited to ethyl acetate, hexamethyldisiloxane, cyclic siloxanes, isopropanol, ethyl alcohol, water, glycols, isododecane, and combinations thereof.

In aspects, an adhesive in accordance with the present disclosure may cure in place on a skin surface to form a tacky adhesive, such that an appliance or device may be securely attached to the surface thereof.

By tacky surface is meant the property of the adhesive surface to bond temporarily to another mating surface by an application of pressure. The tack of an adhesive surface measured using an Instron by Loop Tack test method BS EN 1719:1999. The tacky adhesive according to the present disclosure may have a tack not less than 10 gms of peak force measured per BS EN 1719:1999.

In aspects, an adhesive in accordance with the present disclosure may be configured to cure in place on the skin to form a non-tacky surface. The time taken to form such a surface is commonly referred to as skin-over time. In such aspects, a substantially maximal bond between the adhesive and a mating appliance or device, may be achieved when respective surfaces of the mating bodies (i.e. the adhesive and the appliance or device) are brought into contact prior to the skin-over time of the adhesive. In aspects, the skin over time of the adhesive may take less than 30 minutes, less than 1 hour, less than 2 hours, or the like.

In aspects, a one-part condensation curing skin adhesive according to the present disclosure may include at least one polydimethylsiloxane polymer terminated with acetoxy groups, dibutyltindilaurate catalyst, and an ethyltriacetoxysilane crosslinking agent. In aspects, the polydimethylsiloxane polymer may make up approximately 50-99%, approximately 90-99%, or approximately 96-97% of the adhesive. The catalyst may be present at concentrations of greater than 5 ppm, 20 ppm, 100 ppm, 1000 ppm, or the like of the adhesive. The crosslinking agent may make up approximately 0.1-5%, approximately 1-4%, or approximately 2-3% of the adhesive.

In aspects, a two-part addition curing skin adhesive according to the present disclosure may include at least one divinyl-terminated polydimethylsiloxane, poly(methylhydrogen siloxane-dimethylsiloxane) copolymer, dihydride-terminated polydimethylsiloxane, and a platinum-divinyltetramethyldisiloxane complex based catalyst. In aspects, the divinyl-terminated polydimethylsiloxane may make up approximately 20-99%, approximately, 50-99%, approximately 70-90% of the adhesive. The poly(methylhydrogen siloxane-dimethylsiloxane) copolymer may make up approximately 0.1-20%, approximately 1-15%, approximately 5-10% of the adhesive. The dihydride-terminated polydimethylsiloxane may make up approximately 0.1-30%, approximately 5-20%, approximately, 10-20% of the adhesive. The platinum catalyst complex may make up less than 500 ppm, less than 100 ppm, less than 20 ppm, or the like of the adhesive.

In aspects, an un-cured adhesive according to the present disclosure may have a viscosity suitable for dispensing the liquid onto peri-skin, the viscosity measuring below 500,000 cps, preferably below 100,000 cps as measured per ASTM D1084. The viscosity of the adhesive composition is measured after 24 hours of mixing.

In aspects, a skin compatible curable adhesive in accordance with the present disclosure may be used to protect peri-anal, peri-stomal, peri-wound, and peri-fistula skin.

In aspects, the skin compatible curable adhesive may retain a tacky property even after cure (i.e. retain a pressure sensitive adhesive property after becoming fully cured). In aspects, the skin compatible curable adhesive may revert to a non-tacky property after cure (i.e. such that substantial adhesion to the adhesive may only be attained if the skin, device, or appliance is brought into contact with the adhesive before substantial completion of the cure process).

In aspects, the adhesive may be configured to have a skin-over time on the skin surface within a period of less than 1 hour, preferably of less than 15 minutes, or the like. In aspects, minimally acceptable bond strength may be formed between the skin and the adhesive in less than 24 hours, less than 8 hours, less than 30 minutes, less than 10 minutes, less than 5 minutes, or the like.

In aspects, the adhesive composition may be cured to form a cohesive body. The cohesive body may be substantially insoluble in water, effluent, exudate, urine, feces, excrement, bile salts, blood, or the like for periods of greater than 24 hours, greater than 72 hours, greater than 120 hours, or the like.

In aspects, the skin compatible curable adhesive in accordance with the present disclosure may be configured so as to form a cured-in-place bond to mammalian skin of greater than 0.1 N/in, greater than 1 N/in, greater than 10 N/in, or the like. The same adhesive may be configured to from a cured-in-place bond to a device or appliance in accordance with the present disclosure, the peel strength of the bond being greater than 0.1 N/in, greater than 1.5 N/in, greater than 10 N/in, or the like.

In aspects, an adhesive in accordance with the present disclosure may have a moisture vapor transmission rate (MVTR) in an open cup method (ASTM E96) at 10 mils thick layer of adhesive, greater than 100 grams/square meter/24 hours, greater than 500 grams/square meter/24 hours, greater than 1000 grams/square meter/24 hours, and the like.

The peel strength measurement for the skin compatible curable adhesive in accordance with the present disclosure may be measured using a well-known method, such as with an Instron testing machine. For assessing the peel strength of the bond formed between the adhesive and a skin surface, the adhesive may be applied to an area of skin and allowed to cure. A silicone tape, about 1×5 inches in area, may be adhered gently to the top surface of the curing adhesive. After 24 hours, the silicone tape may be clamped to the Instron and the adhesive and tape peeled off the skin in at an angle of approximately, 90-degrees to the surface of the skin. Similarly, to measure the peel strength of the cured adhesive to the device or appliance, the adhesive of the present disclosure may be applied to the surface of the device or appliance, a silicone tape attached to the surface of the adhesive prior to the skin-over time, and allowed to cure for at least 24 hours. Then the silicone tape, which is bonded to the adhesive, may be peeled off the device or appliance with an Instron at a peeling angle of approximately 90-degrees. The average peel force from multiple pulls may be reported as the peel strength in both cases, for the skin adhesion, and device or appliance adhesion.

In aspects there is provided, a method for securing a device or appliance to a skin surface in accordance with the present disclosure, the method including administering an adhesive composition in accordance with the present disclosure to the skin surface, applying a device or appliance onto the skin surface, and curing the adhesive composition.

In aspects, the method may include forming a skin/adhesive bond between the adhesive composition and the skin, the bond having a peel strength of greater than 0.1 N/in, greater than 1 N/in, greater than 10 N/in, or the like.

In aspects, the method may include forming a device/adhesive bond between the adhesive and the device or appliance, the bond having a peel strength of greater than 25 g/in, greater than 125 g/in, greater than 225 g/in, or the like.

In aspects, the method may include applying a primer to the skin surface. Some non-limiting examples of primers may include water, a mixture of isopropyl alcohol and water, a silane agent, dimethiconol, silicone MQ resin, an interpenetrating network (IPN) forming agent, etc.

In aspects, the method may include mixing one or more components, primers, additives, or the like each in accordance with the present disclosure to form the adhesive composition. In aspects, the mixing may be performed in a static mixer, during delivery of one or more of the components to the skin surface, etc.

According to aspects, there is provided a method for managing a peri-anal, peri-stomal, peri-wound, and peri-fistula skin surface including securing a device or appliance to a skin surface in accordance with a present disclosure using an adhesive composition in accordance with the present disclosure, managing exudate, effluent, and/or bodily waste using the device or appliance, and simultaneously removing the device or appliance, and the adhesive composition from the skin surface.

In aspects, a method in accordance with the present disclosure may include protecting a peri-stomal skin surface from feces while simultaneously securing an ostomy adhesive wafer or ostomy bag to the skin surface, using an adhesive composition in accordance with the present disclosure.

In aspects, a method in accordance with the present disclosure may include forming a seal around a region of peri-stomal skin using an adhesive composition and a device in accordance with the present disclosure.

In aspects, a method in accordance with the present disclosure may include applying a ring of an adhesive composition in accordance with the present disclosure to a peri-wound skin surface surrounding a wound site, applying a dressing to the peri-wound surface and/or the ring of adhesive composition, and forming a cohesive structure between the ring of adhesive composition and the dressing such that the dressing and adhesive composition may be simultaneously removed from the skin surface upon curing of the adhesive composition.

In aspects there is provided, use of a skin compatible curing adhesive in accordance with the present disclosure for peri-wound management.

In aspects, a method in accordance with the present disclosure may include applying a ring of an adhesive composition in accordance with the present disclosure to a peri-fistula skin surface surrounding a fistula, applying a dressing to the peri-fistula surface and/or the ring of adhesive composition, and forming a cohesive structure between the ring of adhesive composition and the dressing such that the dressing and adhesive composition may be simultaneously removed from the skin surface upon curing of the adhesive composition.

In aspects there is provided, use of a skin compatible curing adhesive in accordance with the present disclosure for fistula management.

### EXAMPLES

The following compounds were used in a control example and in examples 1 and 2 according to aspects of the present invention:

Silylated polymer: hydroxy-terminated polydimethylsiloxane DMS-S31 (1000 centistokes) from Gelest, Inc.

Silylated polymer: hydroxy-terminated polydimethylsiloxane RF5000 (5000 centistokes) from Shin Etsu Silicones

Crosslinking agent: Methoxy-terminated polydimethylsiloxane DMS-XM11 from Gelest, Inc.

Crosslinking agent: bis(triethoxysilyl) ethane SIB 1817.0 from Gelest, Inc.

Crosslinking agent: Methyltrimethoxysilane (MTMS) from Sigma Aldrich.

Crosslinking agent: Vinyltrimethoxysilane, SIV 9220.0 from Gelest, Inc.

Titanium catalyst: 2-ethylhexoide tetraoctyltitanate, AKT867 from Gelest, Inc.

Aersoil (hydrophobic fumed silica) R974 and Aerosil (hydrophobic fumed silica) R812 from Evonik Industries.

Hydrophilic additive: Genupectin from C.P. Kelco

### CONTROL EXAMPLE

A composition was prepared comprising DMS-S31: (46% by weight); DMS-XM11: (49.6% by weight); SIB 1817.0: (2.3% by weight); and AKT867: (2.1% by weight).

The DMS-S31, the DMS-XM11 and the SIB 1817.0 were mixed in a plastic container under nitrogen purge. The catalyst AKT867 was then added and the components stirred. The resulting mixture gelled instantly providing a poorly stable 1-part system.

### Examples 1 and 2

### Mix Procedure

The silylated polymer and the cross linking agent MTMS were mixed under vacuum and then ‘fillers’ were added encompassing silica and/or hydrophilic additive under atmospheric pressure. After completing the additions, a Ti catalyst was added under a blanket of nitrogen. After stirring vigorously, vacuum was applied to degas the adhesive mixture. A stable 1-part adhesive mixture was obtained.

### Example 1

Using the above mix procedure, a composition was prepared comprising RF5000 (92.3% by weight); MTMS (2.5% by weight); Aerosil R-974 (4.4% by weight); and Ti Catalyst (0.66% by weight).

### Composition Properties of Example 1

The composition of example 1 formed a stable 1-part adhesive mixture. After 72 hours, various adhesive curing properties were measured.

Skin over: 19.75 minutes—ASTM D-2377-00, where the ASTM D-2377-00 ‘Skin-over time’ is a measure of the time required for a ‘skin’ (a minimum cured layer) to form on the dispensed adhesive such that the skin formation will no longer display a wet surface.

Tack-free: 56.5 minutes (ASTM C-679-09), where the ASTM C-679-09 ‘tack free time’ is the time required for a surface polymerization to be sufficient to resist damage by touch or light contact as identified by the loss of tackiness on the surface.

Durometer: 10 shore A (per ASTM D2240-05) where the ASTM D2240-05 ‘durometer’ hardness test measures the resistance of a rubber specimen 0.5-inch thick minimum to a spring loaded needle.

### Example 2

Using the above mix procedure, a composition was prepared comprising DMS-S31 (85.2% by weight); Aerosil 812 (3.3% by weight); Genupectin (1.65% by weight); MTMS (8.3% by weight); AKT865 (0.99% by weight); and 5IV9220.0 (0.56% by weight).

### Composition Properties of Example 2

The composition of example 2 was formed as a stable 1-part adhesive mixture. The composition was observed to be tack-free less than 30 minutes following the end of the mix procedure.

It will be appreciated that additional advantages and modifications will readily occur to those skilled in the art. Therefore, the disclosures presented herein and broader aspects thereof are not limited to the specific details and representative embodiments shown and described herein. Accordingly, many modifications, equivalents, and improvements may be included without departing from the spirit or scope of the general inventive concept as defined by the appended claims and their equivalents.

1. A skin compatible curable adhesive to protect a region of a skin surface, for the treatment or management of one or more medical conditions, the adhesive comprising: at least one silylated polymer, silylated macromer, silylated monomer, or a combination thereof; at least one curing catalyst; and at least one crosslinking agent.

2. The skin compatible curable adhesive in accordance with claim 1, wherein the adhesive comprises a pre-cured state and a post-cured state, wherein the post cured state is tacky to the touch with a loop tack force greater than 0.1 N/in.

3. The skin compatible curable adhesive in accordance with claim 1, wherein the adhesive comprises a pre-cured state and a post-cured state, wherein the post cured state is non-tacky to the touch with a loop tack force of less than 0.1 N/in.

4. The skin compatible curable adhesive in accordance with claim 3, wherein the adhesive is configured to form a cohesive bond to a surface of a device applied thereto only while in the pre-cured state.

5. The skin compatible curable adhesive in accordance with claim 2, wherein the pre-cured form has a viscosity less than 500,000 cps, less than 250,000 cps, or less than 100,000 cps.

6. The skin compatible curable adhesive in accordance with claim 2, wherein the adhesive is configured to be applied to the region of skin in the pre-cured state, and to transition to the post cured state on the skin, the adhesive in the post cured state forming a bond to the skin, the peel strength thereof in the range of 0.1 to 10 N/in.

7. The skin compatible curable adhesive in accordance with claim 2, wherein the adhesive is configured to transition from the pre-cured state to the post-cured state upon application to the skin, the transition taking less than 7 days, less than 24 hours, less than 1 hour, less than 30 minutes, or less than 15 minutes.

8. The skin compatible curable adhesive in accordance with claim 2, wherein the adhesive is configured to undergo a curing reaction to transition between the pre-cured state and the post-cured state, the curing reaction initiated via exposure of the adhesive to moisture, ultraviolet light, visible light, infrared radiation, heat, oxygen, or a combination thereof.

9. The skin compatible curable adhesive in accordance with claim 1, wherein the silylated polymer, silylated macromer, or silylated monomer comprises at least two or more pendant or terminal silylated functional groups selected from vinyl-silyl, hydride-silyl, alkoxysilyl, aryloxysilyl, acryloxysilyl, alkyloximinosilyl, hydroxyl-silyl, amino-silyl, halo-silyl, or a combination thereof.

10. The skin compatible curable adhesive in accordance with claim 1 comprising one or more of divinyl-terminated polydimethylsiloxane, dihydride-terminated polydimethylsiloxane, polymethylhydrogensiloxane-polydmethylsiloxane copolymer, acetoxy-terminated polydimethylsiloxane, silylated polyether, silylated polyurethane, silylated polyolefin, silylated polyester, silylated acrylic, or a combination thereof.

11. The skin compatible curable adhesive in accordance with claim 1, comprising a silicone polymer, silicone macromer, silicone monomer, or a combination thereof.

12. The skin compatible curable adhesive in accordance with claim 1, comprising one or more hydrophilic additive.

13. The skin compatible curable adhesive in accordance with claim 12, wherein the hydrophilic additive comprises one or more of sodium carboxymethylcellulose (NaCMC), pectin, gelatin, chitosan, polyvinyl pyrrolidone and its copolymers, polyvinyl alcohol and its copolymers, polyacrylic acid, its copolymers and salts, nanoclays, cellulosic fibers, starch, polyacrylamide and its copolymers, poly(N-alkylacrylamide) and its copolymers, polyethylene glycol and its copolymers, polyethyleneoxide, unmodified and modified silica, poly(maleic anhydride) and its copolymers, or combinations thereof.

14. The skin compatible curable adhesive in accordance with claim 1 wherein the catalyst is one or more of tin-based, titanium-based, zinc-based, or copper-based, or platinum based.

15. The skin compatible curable adhesive in accordance with claim 1 comprising one or more of dibutyltindilaurate, bis(2-ethylhexanoate)tin, dioctyltindilaurate, tetraoctyltitanate, titatium diisopropoxide(bis-2,4-pentanedionate), tetrakis(trimethylsiloxy)titanium, platinum-divinyltetramethyldisiloxane complex, platinum-cylcovinylmethylsiloxane complex, or combinations thereof.

16. The skin composition in accordance with claim 1 wherein the crosslinking agent comprises ethyltriacetoxysilane, methyltriacetoxysilane, vinyltriacetoxysilane, tetraethoxysilane, methyltris(methylethylketoximino)silane, bis(N-methybenzamido)ethoxymethyl-silane, vinyl-MQ resin, hydride-MQ resin, hydroxyl-MQ resin, polymethylhydrogensiloxane-dimethylsiloxane copolymer, polymethylvinylsiloxane-dimethylsiloxane copolymer, or a combination thereof.

17. The skin compatible curable adhesive in accordance with claim 2, wherein the adhesive provided in the post-cured state is insoluble in water.

18. The skin compatible curable adhesive in accordance with claim 1, wherein the adhesive is configured to be stored as a single component system.

19. The skin compatible curable adhesive in accordance with claim 1, wherein the adhesive is configured to be stored as a two-part system, the two-parts mixable to form the pre-cured state in accordance with claim 2.

20. The skin compatible curable adhesive in accordance with claim 1 comprising the silylated polymer at 80 to 98% by weight; the at least one curing catalyst at trace to 4% by weight; and the at least one crosslinking agent at trace to 15% by weight.

21. The skin compatible curable adhesive in accordance with claim 20 comprising the silylated polymer at 90 to 96% by weight; the at least one curing catalyst at trace to 2.0% by weight; the at least one crosslinking agent at 1.0 to 3.5% by weight and a filler comprising any one or a combination of a silica, a hydrophobic fumed silica and/or a hydrophilic additive at 1 to 10% by weight.

22. The skin compatible curable adhesive as claimed in claim 1 comprising the silylated polymer at 82 to 89% by weight; at least one curing catalyst at trace to 5% by weight; a first type of crosslinking agent at 6 to 10% by weight; a second type of crosslinking agent at trace to 2% by weight; a pectin at trace to 5% by weight and a filler comprising any one or a combination of a silica, a hydrophobic fumed silica and/or a hydrophilic additive at 1 to 10% by weight.

23. The skin compatible curable adhesive in accordance with claim 1 wherein the region of skin is a pen-anal, peri-stomal, pen-wound, or peri-fistula skin.

24. Use of a skin compatible curable adhesive in accordance with claim 1 to protect pen-anal, peri-stomal, pen-wound, or peri-fistula skin.

25. A method for protecting and/or managing a peri-skin surface on a body comprising:
administering an adhesive composition to the peri-skin surface, the adhesive comprising: at least one silylated polymer, silylated macromer, silylated monomer, or a combination thereof; at least one curing catalyst; and at least one crosslinking agent;
applying a device to the adhesive composition; and
curing the adhesive composition.

26. The method in accordance with claim 25, comprising applying a primer to, cleaning, and/or preparing the peri-skin prior to the administering of the adhesive composition.

27. The method in accordance with claim 25, comprising mixing two or more parts to form the adhesive composition prior to the step of administering the adhesive composition to the peri-skin.

28. The method in accordance with claim 27, wherein the mixing is performed with a static mixer, the two or more parts stored in compartments, and delivered to the peri-skin via the static mixer.

29. The method in accordance with claim 25, comprising forming a skin-side bond between the peri-skin and the adhesive composition, the peel strength thereof in the range of 0.1 to 10 N/in.

30. The method in accordance with claim 25, comprising forming a device-side bond between the device and the adhesive composition, the peel strength of which is greater than 15 g/in, greater than 125 g/in, or greater than 225 g/in.

31. The method in accordance with claim 25, maintaining the adhesive composition against the peri-skin for greater than 24 hours, greater than 48 hours, greater than 72 hours.

32. The method in accordance with claim 25, comprising simultaneously removing the device and the adhesive composition from the peri-skin.

33. The method in accordance with claim 32, wherein the cohesive bond formed between the device and the adhesive composition is maintained after removed from the peri-skin.

34. The method in accordance with a claim 25, comprising administering the adhesive composition in a ring-like shape onto the peri-skin.

35. The method in accordance with claim 25, wherein the peri-skin is a peri-anal, peri-stomal, peri-wound, or peri-fistula skin.

36. The method in accordance with claim 25, wherein the device is a wound dressing, a surgical drape, a film, foam, an ostomy adhesive wafer, or an ostomy bag.

37. The method in accordance with claim 25, wherein the cured adhesive composition has a hardness value below Shore 50 A, preferably below 40 A, more preferably below 35 A.
